(19) United States
(12) Patent Application Publication
(10) Pub. No.: US 2022/0087766 A1
(43) Pub. Date: Mar. 24, 2022

(54) ANESTHESIA PACKAGE

(71) Applicant: John Hagopian, Mentor, OH (US)

(72) Inventor: John Hagopian, Mentor, OH (US)

(21) Appl. No.: 17/030,575

(22) Filed: Sep. 24, 2020

(51) Int. Cl. A61B 50/30; A61B 50/33

(52) U.S. Cl. CPC A61B 50/30; A61B 2050/3008; A61B 50/33

(57) ABSTRACT

A pre-sterilized, anesthesia kit for use with an anesthesia machine includes a re-usable, molded, thermoformed container formed from plastic materials to a desired shape, size, and topography. The kit further includes an anesthesia package including a unitary, multi-compartment tray formed with appropriate dividers and compartments for storing various medical instruments and wrapped within an inner package wrap. The anesthesia package is disposed in the molded, thermoformed container. A sheet of paper having adhesive on one side and removably attached to the molded, thermoformed container. A plastic sleeve with a cardboard corrugation stored between the sheet of paper and the anesthesia package.

### TECHNICAL FIELD OF THE INVENTION

The present invention relates to the field of sterile packaging of components to be used in certain medical operative procedures. More specifically, the invention relates to the packaging, in a single package, of various components used in anesthesia procedures.

### BACKGROUND OF THE INVENTION

Medical practitioners work together through an orchestrated sequence of steps to ensure that best practices for the delivery of a particular procedure is attained in accordance with a particular standard of patient care. For example, most surgical and medical procedures follow a logical sequence of steps that may involve any combination of assessment, diagnosis, imaging, preparation, procedure and follow-up.

Importantly, in many scenarios, while best practices may suggest that preferred procedures be followed, the particulars of an emergency medical condition may limit the ability of the medical team to complete the preferred steps either because of time constraints and/or the availability of equipment and/or tools to enable those steps.

For example, in the case of assessing and treating patients requiring anesthesia, a number of different medical tools and medicines are initially selected and prepared in case of an emergency. These include but are not limited to a tongue depressor, EKG leads, lubricant, tape, injection syringes, a pre-formed cotton bite-block, an oral airway, several alcohol prep pads, and several 4x4″ cotton gauze sponges. It's very important they everything in the operating field is kept sterile. Therefore, at the end of a procedure, almost everything that is put in place for possible use is either discarded or left for another patient assuming that it is still wrapped in a sterile package. It's understood that any time saved during the procedures to assess, diagnose, image and treat a patient may have significant effects on the status of the patient.

It will be clear from the above description that each of the medical procedure components used in an anesthesia procedure, including tongue depressor, EKG leads, lubricant, tape, and injection syringes, must all be pre-sterilized before or during packaging and, most importantly, must be maintained in a sterile condition until used. The conventional approach to the problem of ensuring sterility in each of the components, some of which are typically necessary in any procedure, is to store and ship the components in sterilized packaging.

When the decision is made to initiate a surgical treatment, a number of factors will come into play that must all be managed to enable treatment in what is a complex emergency situation. These factors include the time of day (may be after regular staffing hours), the assembly of different teams and the requirements for patient monitoring, anesthesia, consent and patient preparation for the procedure.

Further, particularly when such procedures are being conducted as an emergency outside normal staffing hours, different members of the team may reach the operating theatre at slightly different times. Depending on the time of arrival and the particular training of the team members, it may be difficult for certain team members to initiate the preparatory steps such as gathering the medical equipment needed for the procedure due to the lack of training to assemble and/or undertake such steps. Any delays in preparing the patient and/or the equipment will result in delays in initiating and completing the medical procedure itself. Moreover, medical teams do not want to be assembling or looking for equipment in the middle of the night when the procedure may be required. In addition, it may be desired that the procedure is performed at a hospital or treatment facility where the procedure is not routinely performed with the result being that a physician and/or the medical team may not be familiar with the layout of an operating room and/or the storage locations of the equipment. As such, there is a need for the medical equipment to be transportable in a convenient form so as to minimize time delays under these conditions.

Particularly of interest are improvements in the safety of the procedure and kits as well as any timesaving devices or steps.

### SUMMARY OF THE INVENTION

In accordance with the present invention there is disclosed a pre-sterilized, anesthesia kit for use with an anesthesia machine. The kit comprises in combination a re-usable, molded, thermoformed container formed from plastic materials to a desired shape, size, and topography. The kit further includes an anesthesia package including a unitary, multi-compartment tray formed with appropriate dividers and compartments for storing various medical instruments and wrapped within an inner package wrap. The anesthesia package is disposed in the molded, thermoformed container. A sheet of paper having adhesive on one side and removably attached to the molded, thermoformed container. A plastic sleeve with a cardboard corrugation stored between the sheet of paper and the anesthesia package.

Further in accordance with the present invention there is disclosed a pre-sterilized, anesthesia kit for use with an anesthesia machine. The kit comprises a re-usable, molded, thermoformed container formed from plastic materials to a desired shape, size, and topography. An anesthesia package including a unitary, multi-compartment tray formed with appropriate dividers and compartments for storing various medical instruments selected from the group consisting of tongue depressors, EKG leads, lubricants, tape, injection syringes, and intubation medical tools is wrapped within an inner package wrap formed of several layers of a surgical towel. The anesthesia package is disposed in the molded, thermoformed container. A sheet of paper having adhesive on one side and removably attached to the molded, thermoformed container. A plastic sleeve with a cardboard corrugation stored between the sheet of paper and the anesthesia package. A sheet of instructions, to indicate the correct position of the anesthesia package, with adhesive on one side is removably adhered to an outer facing surface of the surgical towel. A transparent flexible plastic sheet placed against the surgical towel and wrapped with the towel is disposed on the anesthesia machine.

### BRIEF DESCRIPTION OF THE DRAWINGS

The structure, operation, and advantages of the present invention will become further apparent upon consideration of the following description taken in conjunction with the accompanying figures (FIGS.). The figures are intended to be illustrative, not limiting. Certain elements in some of the figures may be omitted, or illustrated not-to-scale, for illustrative clarity. The cross-sectional views may be in the form of “slices”, or “near-sighted” cross-sectional views, omitting certain background lines which would otherwise be visible in a “true” cross-sectional view, for illustrative clarity.

In the drawings accompanying the description that follows, both reference numerals and legends (labels, text descriptions) may be used to identify elements. If legends are provided, they are intended merely as an aid to the reader, and should not in any way be interpreted as limiting.

FIG. 1 is a perspective view of a pre-sterilized, anesthesia package kit in a wrapped tray held in a box, in accordance with the present invention.

FIG. 2 is a perspective view of pre-sterilized, anesthesia package kit disposed in a tray located on the top of an anesthesia machine, in accordance with the present invention.

FIG. 3 is a perspective view of the pre-sterilized, anesthesia package kit in an uncovered tray with a portion of package wrap unfolded on the top of the anesthesia machine, in accordance with the present invention.

FIG. 4 is a top perspective view of the pre-sterilized, anesthesia package kit in an uncovered tray with a portion of package wrap completely unfolded on the top of an anesthesia machine and having various medical instruments disposed on the package wrap, in accordance with the present invention.

FIG. 5 is a top perspective view of the pre-sterilized, anesthesia package kit in an uncovered tray with a portion of package wrap completely unfolded on the top of an anesthesia machine and having various medical instruments disposed on the package wrap, in accordance with the present invention.

FIG. 6 is a front, perspective view of the arm of an anesthesia machine covered with a plastic sleeve and having a bag mounted thereto, in accordance with the present invention.

### DETAILED DESCRIPTION OF THE INVENTION

The invention will now be described in detail with reference to the foregoing drawings wherein like numerals are used to identify like parts.

The structure, operation, and advantages of the present invention will become further apparent upon consideration of the following description taken in conjunction with the accompanying figures (FIGS.). The figures are intended to be illustrative, not limiting. Certain elements in some of the figures may be omitted, or illustrated not-to-scale, for illustrative clarity. The cross-sectional views may be in the form of “slices”, or “near-sighted” cross-sectional views, omitting certain background lines which would otherwise be visible in a “true” cross-sectional view, for illustrative clarity.

In the drawings accompanying the description that follows, both reference numerals and legends (labels, text descriptions) may be used to identify elements. If legends are provided, they are intended merely as an aid to the reader, and should not in any way be interpreted as limiting.

The present invention relates to a pre-sterilized, anesthesia kit 10 which comprises a tray 12 containing a pre-sterilized, anesthesia package 14 including a molded, thermoformed container 16 formed from plastic materials to a desired shape, size, and topography. Formed plastic materials include, for example, thermoformed and injection molded polymers. Suitable plastic materials for use in this invention include, for example, polyethylene, polyester, polycarbonate, polyethylene terephthlate, polystyrene, and polyvinylchloride. An anesthesia package 14 comprising a tray 16 wrapped in surgical towel 18, as discussed in more detail later, is formed with appropriate dividers and compartments for storing various medical instruments or supplies, such as tongue depressors, EKG leads, lubricants, tape, an injection syringe such as a ten cc injection syringe, a pre-formed cotton bite-block, an oral airway, several alcohol prep pads, several cotton gauze sponges and intubation medical tools, such as, as laryngoscopes, endotracheal tubes, and laryngeal mask airways.

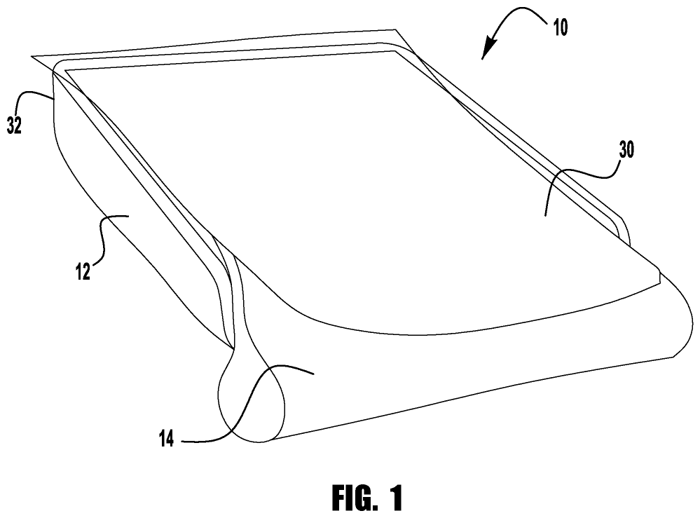

When the pre-sterilized, anesthesia kit 10 is complete and ready for being shipped in commerce as shown in FIG. 1, a sheet 30 of paper having adhesive on one side of the pre-sterilized anesthesia package 14 which is disposed in a transfer container 12. The sheet 30 is pulled up and over the back end 32 of the transfer container 12 to expose the pre-sterilized, anesthesia package 14. A plastic sleeve 82 with a cardboard or plastic corrugation 84, as discussed in more detail below can be stored between the sheet 30 and the anesthesia package 14. The sleeve 82 is initially removed and placed in a handy location for it to be employed as discussed herein after.

After the pre-sterilized anesthesia package 14 has been removed from the transfer container 12, the transfer container can be put aside and used to store various medical devices after they've been used for re-sterilization or any other desired purpose.

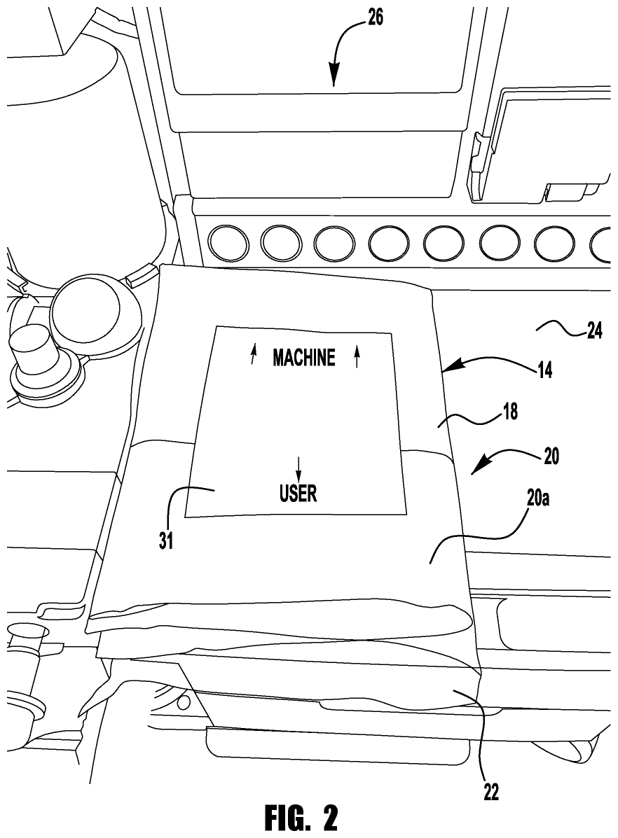

As shown in FIG. 2, the pre-sterilized anesthesia package 14 includes the tray 16 wrapped with several layers 20 of a surgical towel 18 which is typically formed of cotton so as to be fast, effective, and provide an easy solution for wide range of uses in any medical facility. The surgical towel is sterile, highly absorbent and virtually lint free. Their super absorbent quality and soft texture make them work excellently well for medical treatment. 100% cotton provides maximum absorbency and uniform consistency. The towels, typically blue, feature untreated, absorbent cotton material designed for extra comfort and strength. The surgical towels provide a cost-effective way to reduce the infection risks.

Next, the pre-sterilized anesthesia package 14, after being removed from the container 12, can be placed on the top surface 24 of the anesthesia machine 26. A sheet of instructions 31 with adhesive on one side can be removably adhered on the outer facing surface of the top layer 20a of the towel 18 wrapped around the anesthesia package 14. The sheet of instructions 31 can have printed matter to indicate the correct position of the pre-sterilized, anesthesia package 14 on the top surface 24 of the anesthesia machine 26.

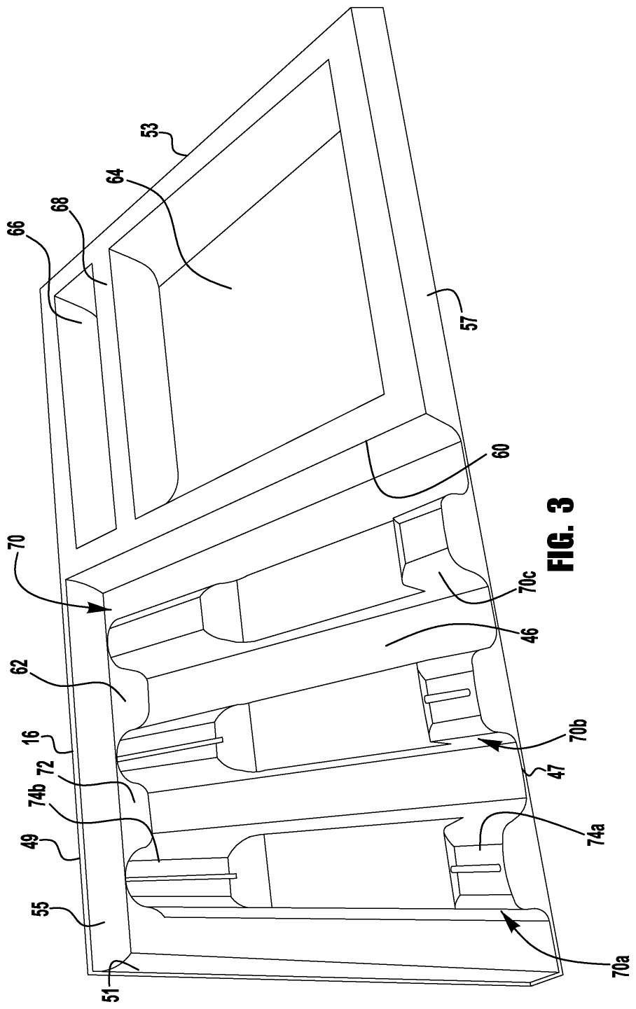

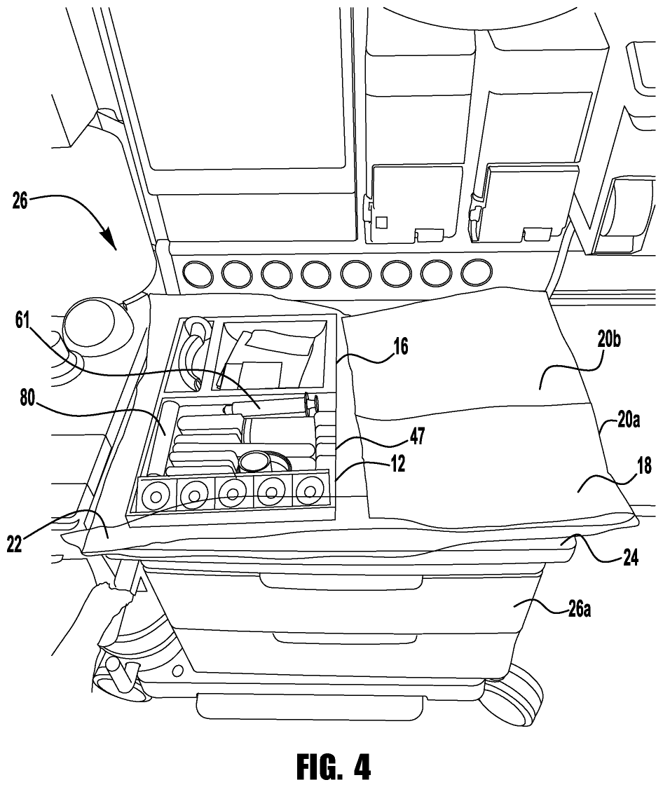

A transparent flexible plastic sheet 22 is placed against the surgical towel 18 and wrapped with the towel as shown in FIG. 2. Initially, the pre-sterilized anesthesia package 14 is placed on the top surface 24 of an anesthesia machine 26. At this stage, the transparent flexible plastic sheet 22 is disposed directly on the top surface 24 of the anesthesia machine 26. Next, the top layer 20a of the towel 18 is unfolded from the molded tray 16 and placed against the top surface 24 of the anesthesia machine 26 as shown in FIG. 3. Note that the transparent flexible plastic sheet 22 is disposed directly against the top surface 24 of the anesthesia machine 26. Then, assuming the top surface 24 of the anesthesia machine is long enough, the layer 20b of the towel 18 is again unfolded from the molded tray 16 and placed against the top surface 24 of the anesthesia machine 26 as shown in FIG. 4, with the transparent flexible plastic sheet 22 disposed between the towel 18 and the top surface 24 of the anesthesia machine 26. The transparent flexible plastic sheet 22 extending outward away from the front 26a of the anesthesia machine 26 from the towel 18 can fold over the front 26a of the anesthesia machine 26 so that various controls (not shown) located on the front of the machine are visible and accessible to the medical practitioner.

When the pre-sterilized, anesthesia package unit 10 is complete and ready for being shipped in commerce as shown in FIG. 1, a sheet 30 of paper having adhesive on one side of the pre-sterilized anesthesia package 14 which is disposed in a transfer container 12. The sheet 30 is pulled up and over the back end 32 of the container 12 to expose the pre-sterilized, anesthesia package 14. A plastic sleeve 82 with a cardboard or plastic corrugation 84, as discussed in more detail below can be stored between the sheet 30 and the anesthesia package 14. The sleeve 82 is initially removed and placed in a handy location for it to be employed as discussed herein after.

Next, the pre-sterilized anesthesia package 14 can be removed from the container 12 and placed on the top surface 24 of the anesthesia machine 26. A sheet 31 of paper with adhesive on one side can be removably adhered on the outer facing surface of the top layer 20a of the towel 18. The sheet 31 can have printed matter to indicate the correct position of the pre-sterilized, anesthesia package 14 on the top surface 24 of the anesthesia machine 26.

Once the anesthesia package 14 has been unfolded on the top surface 24 of the anesthesia machine 26 as shown in FIG. 4, the inner package comprises a set-up tray 16, for example of molded or thermo-formed plastic having essentially two compartments. Some materials suitable for such constructions include styrene, resins, rubber, or other pliant compounds. In other embodiments, materials such as acrylonitrile butadiene styrene (ABS), polycarbonate (PC) and polycarbonate-ABS can be used. It will be clear to those of ordinary skill in the art having the benefit of this disclosure that other materials and manufacturing methods can be used.

The set-up tray 16, as shown in FIG. 3, has a base surface 46 that extends from a first side 47 across a length of the base surface to a distal second side 49. While the length of the base surface 46 can vary based upon application, in one embodiment the length is between 200 millimeters and 205 millimeters. The width of the base surface 46 extends from a third side 51 across a width of the base surface to a distal fourth side 53. While the width of the base surface 46 can vary based upon application, in one embodiment the width is between 300 millimeters and 305 millimeters. Other lengths and widths for the base surface are also within the terms of the invention.

A stop wall 55 is present on the second side 49, the third side 51, the fourth side 53 and a portion of the fifth side 57. The stop wall 55 is configured as a raised rim that extends upwardly from the base surface 46 and passes along the edges of the second side 49, the third side 51 and the fourth side 53 and the fifth side 57. An intermediate raised rim 60 extends from the first side 47 across a length of the base surface 46 to a distal second side 49. The intermediate raised rim 60 divides the tray 16 into a first compartment 62 and second and third adjacent compartments 64 and 66, respectively. The second and third adjacent compartments 64 and 66 are divided by a raised rim 68 extending between the intermediate raised rim 60 and the fourth side 53.

Typically, the first compartment 62 is equipped with appropriate dividers 70, such as three dividers 70a, 70b, and 70c (70a-70c) which extend from the first side 47 to a slot 72. The slot 72 is located between the stop wall 55 forming the third side wall 51, the second side 49 and the intermediate raised rim 60 and the end portions of the three dividers 70a, 70b, and 70c. Each of the dividers 70a-70c has two spaced portions 74a and 76a which extend upward from the base surface 46 and are configured to be flexible or pliant to grip medical implements inserted on the portion of the base surface 46 between the sidewall 51 and the divider 74a, between the divider 74a and the divider 74b, between the divider 74b and the divider 74c, and the divider 74c and the intermediate raised rim 60.

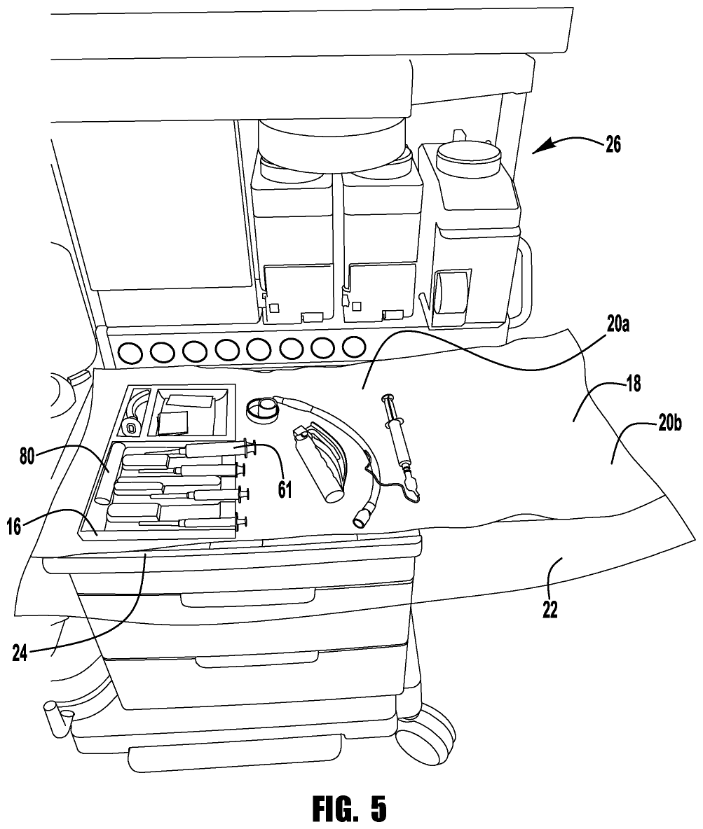

For example, as shown in FIGS. 3 and 4 and 5, the bottom surface 46 between the first side 47 to a slot 72 which is between the stop wall 55 forming the side wall 51, the second side 49 and the intermediate raised rim 60 are open at the end 47 of the tray and are each adapted for supporting the barrel of a drug syringe 61 with a needle. The bottom surface 46 opens at one side 47 of the tray and allows the barrel flange of the syringe to rest against the sidewalls of the dividers 74a-74c and the first sidewall 51 and the raised rim 60. In practice, the syringes with a needle and placed between the dividers after the anesthesia package 14 is opened so that the appropriate syringes with the correct drugs can be selected depending on the circumstances of the particular patient.

The dividers 74a-74c can also be used for storing various medical instruments or supplies, such as scrubbing or cleansing elements. The slot 72 can be used to store a pre-formed cotton bite block 80. Other medical devices such as electrodes and tongue depressors can also be stored between dividers 74a-74c. The second compartment 64 can be used to store different types of tape for securing a tube to the face of the patient or taping the eyes closed. Other types of medical supplies such as ointments can also be stored in the second compartment 64.

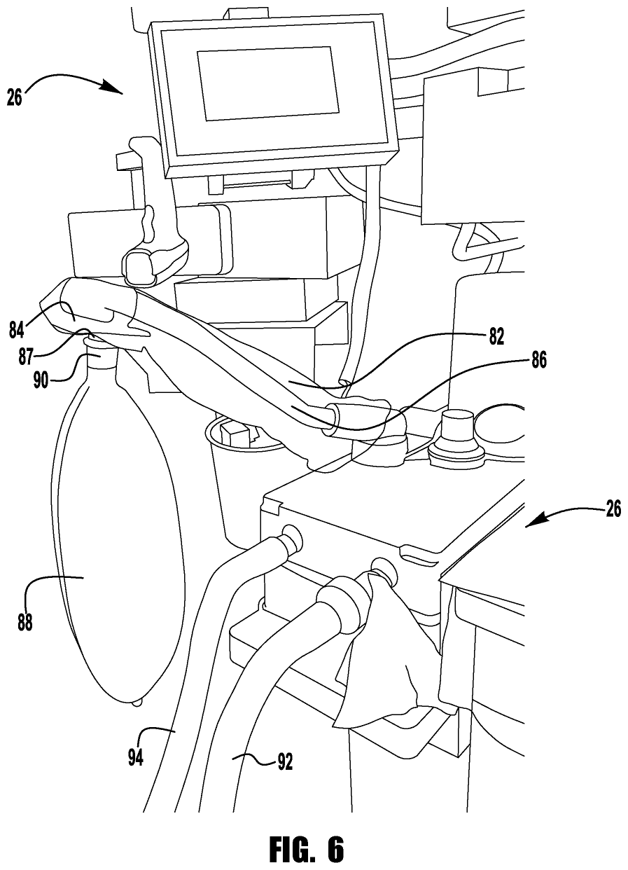

When the pre-sterilized, anesthesia package 14 is being opened, see FIG. 6, a plastic sleeve 82 with a cardboard or plastic corrugation 84 can be disposed on the top of the sheet 30 of paper having adhesive on one side. The plastic sleeve 82 can initially be pulled onto the arm 86 of the anesthesia machine so that the plastic sleeve fits around a protruding gas outlet pipe 87. Then, a bag 88 is attached at one end 90 to the outlet pipe. The plastic sleeve 82 protects the arm 86 from being contaminated when a medical person rests a face mask on the arm which is often the case at the beginning and end of the medical procedure.

When the medical procedure is completed, the face mask (not shown) and the associated tubing 92 and 94, the bag 88, and other components which have been used and need to be disposed of can be wrapped up in the tray or container 16 with the transparent flexible plastic sheet 22 and the towel 18 so that it can easily be disposed of. The syringes 61 are normally disposed of in a sharp object storage box. Other instruments that are to be sterilized and packaged again, can be placed in the tray 12.

It will be seen that when the packages of the invention are prepared and used as described above, the internal sterility of the packages and their contents is maintained from final package sealing until the packages are opened for use. Likewise, the surfaces of the anesthesia machine is also kept sterile.

Although the invention has been shown and described with respect to a certain preferred embodiment or embodiments, certain equivalent alterations and modifications will occur to others skilled in the art upon the reading and understanding of this specification and the annexed drawings. In particular regard to the various functions performed by the above described components (assemblies, devices, etc.) the terms (including a reference to a “means”) used to describe such components are intended to correspond, unless otherwise indicated, to any component which performs the specified function of the described component (i.e., that is functionally equivalent), even though not structurally equivalent to the disclosed structure which performs the function in the herein illustrated exemplary embodiments of the invention. In addition, while a particular feature of the invention may have been disclosed with respect to only one of several embodiments, such feature may be combined with one or more features of the other embodiments as may be desired and advantageous for any given or particular application.

We claim:

1. A pre-sterilized, anesthesia kit for use with an anesthesia machine comprising in combination:
a re-usable, molded, thermoformed container formed from plastic materials to a desired shape, size, and topography;
an anesthesia package including a unitary, multi-compartment tray formed with appropriate dividers and compartments for storing various medical instruments and wrapped within an inner package wrap, said anesthesia package disposed in the molded, thermoformed container;
a sheet of paper having adhesive on one side and removably attached to the molded, thermoformed container; and
a plastic sleeve with a cardboard corrugation stored between the sheet of paper and the anesthesia package.

2. The pre-sterilized, anesthesia kit of claim 1 wherein the molded, thermoformed container is formed from a plastic material selected from the group consisting of polyethylene, polyester, polycarbonate, polyethylene terephthlate, polystyrene, and polyvinylchloride.

3. The pre-sterilized, anesthesia kit of claim 1 wherein the multi-compartment tray can store various medical instruments selected from the group consisting of tongue depressors, EKG leads, lubricants, tape, and injection syringes, a pre-formed cotton bite-block, an oral airway, several alcohol prep pads, several cotton gauze sponges and intubation medical tools.

4. The pre-sterilized, anesthesia kit of claim 3 wherein the inner package wrap is formed of several layers of a surgical towel.

5. The pre-sterilized, anesthesia kit of claim 4 further including a sheet of instructions with adhesive on one side to be removably adhered to an outer facing surface of the surgical towel.

6. The pre-sterilized, anesthesia kit of claim 5 wherein the sheet of instructions can have printed matter to indicate the correct position of the anesthesia package on a top surface of the anesthesia machine.

7. The pre-sterilized, anesthesia kit of claim 6 wherein a transparent flexible plastic sheet is placed against the surgical towel and wrapped with the towel.

8. The pre-sterilized, anesthesia kit of claim 1 wherein the multi-compartment tray is formed from a material selected from the group consisting of styrene, resins, rubber, acrylonitrile butadiene styrene (ABS), polycarbonate (PC) and polycarbonate-ABS.

9. The pre-sterilized, anesthesia kit of claim 8 wherein the multi-compartment tray has a base surface that extends from a first side across a length of the base surface to a distal second side, and a width of the base surface extends from a third side across a width of the base surface to a distal fourth side.

10. The pre-sterilized, anesthesia kit of claim 9 wherein a stop wall is present on the second side, the third side, the fourth side and a portion of a fifth side.

11. The pre-sterilized, anesthesia kit of claim 10 wherein the stop wall is configured as a raised rim that extends upwardly from the base surface and passes along edges of the second side, the third side, the fourth side, and the fifth side.

12. The pre-sterilized, anesthesia kit of claim 11 wherein an intermediate raised rim extends from the first side across a length of the base surface to a distal second side, said intermediate raised rim dividing the tray into a first compartment and second and third adjacent compartments.

13. The pre-sterilized, anesthesia kit of claim 12 wherein the second and third adjacent compartments are divided by a raised rim extending between the intermediate raised rim and the fourth side.

14. The pre-sterilized, anesthesia kit of claim 13 wherein the first compartment is equipped with three dividers which extend from the first side to a slot which is between the stop wall forming the side wall, the second side and the intermediate raised rim.

15. The pre-sterilized, anesthesia kit of claim 14 wherein each of the three dividers has two spaced portions which extend upward from the base surface and are configured to be flexible to grip medical implements.

16. The pre-sterilized, anesthesia kit of claim 1 wherein the plastic sleeve can be pulled onto an arm of the anesthesia machine so that the plastic sleeve fits around a protruding gas outlet pipe.

17. A pre-sterilized, anesthesia kit for use with an anesthesia machine comprising in combination:
a re-usable, molded, thermoformed container formed from plastic materials to a desired shape, size, and topography;
an anesthesia package including a unitary, multi-compartment tray formed with appropriate dividers and compartments for storing various medical instruments selected from the group consisting of tongue depressors, EKG leads, lubricants, tape, an injection syringe, a pre-formed cotton bite-block, an oral airway, several alcohol prep pads, several cotton gauze sponges and intubation medical tools, and wrapped within an inner package wrap formed of several layers of a surgical towel, said anesthesia package disposed in the molded, thermoformed container;
a sheet of paper having adhesive on one side and removably attached to the molded, thermoformed container;
a plastic sleeve with a cardboard corrugation stored between the sheet of paper and the anesthesia package;
a sheet of instructions, to indicate the correct position of the anesthesia package, with adhesive on one side to be removably adhered to an outer facing surface of the surgical towel; and
a transparent flexible plastic sheet placed against the surgical towel and wrapped with the towel to be disposed on the anesthesia machine.

18. The pre-sterilized, anesthesia kit of claim 17 wherein the multi-compartment tray has a base surface that extends from a first side across a length of the base surface to a distal second side, and a width of the base surface extends from a third side across a width of the base surface to a distal fourth side.

19. The pre-sterilized, anesthesia kit of claim 18 wherein a stop wall is configured as a raised rim that extends upwardly from the base surface and passes along edges of the second side, the third side, the fourth side, and the fifth side.

20. The pre-sterilized, anesthesia kit of claim 19 wherein an intermediate raised rim extends from the first side across a length of the base surface to a distal second side, said intermediate raised rim dividing the tray into a first compartment and second and third adjacent compartments.
